(19) United States
(12) Patent Application Publication
(10) Pub. No.: US 2022/0265653 A1
(43) Pub. Date: Aug. 25, 2022

(54) PHARMACEUTICAL COMPOSITION OF IMATINIB

(71) Applicant: INTAS PHARMACEUTICALS LTD., Thaltej, Gujarat Ahmedabad (IN)

(72) Inventors: Jwalant Vijaybhai DESAI, Sanand Ahmedabad, Gujarat (IN); Mayank SAXENA, Sanand Ahmedabad, Gujarat (IN); Ashutosh JAMLOKI, Sanand Ahmedabad, Gujarat (IN); Venkataramana NAIDU, Sanand Ahmedabad, Gujarat (IN)

(73) Assignee: INTAS PHARMACEUTICALS LTD., Thaltej, Gujarat Ahmedabad (IN)

(21) Appl. No.: 17/627,014

(22) PCT Filed: Jul. 15, 2020
(86) PCT No.: PCT/IB2020/056641

§ 371 Date: Jan. 13, 2022

(30) Foreign Application Priority Data
Jul. 15, 2019 (IN) 201921028370

(51) Int. Cl. A61K 31/506; A61K 9/16

(52) U.S. Cl. CPC A61K 31/506; A61K 9/1694

(57) ABSTRACT

A pharmaceutical composition having imatinib or its pharmaceutically acceptable salt thereof and one or more pharmaceutical acceptable excipients in powder form, which can be reconstituted with a diluent just before administration. Also disclosed is a process for the preparation of the composition and its use in a method for the treatment of cancer in pediatric patients.

### RELATED APPLICATIONS

This application is related to Indian Provisional Application No. 201921028370 filed on 15 Jul., 2019 and is incorporated herein in its entirety.

### FIELD OF THE INVENTION

The present invention relates to a pharmaceutical composition comprising imatinib or its pharmaceutically acceptable salt thereof and one or more pharmaceutical acceptable excipients in powder form, which can be reconstituted with a diluent just before administration. Further, the present invention provides process for the preparation of the said composition and its use for the treatment of cancer in pediatric patients.

### BACKGROUND OF THE INVENTION

Imatinib is a protein-tyrosine kinase inhibitor which is chemically known 4-[(4-methylpiperazin-1-yl)methyl]-N-(4-methyl-3-{[4-(pyridin-3-yl)pyrimidin-2-yl] amino}phenyl)benzamide and molecular formula is C₂₉H₃₁N₇O.

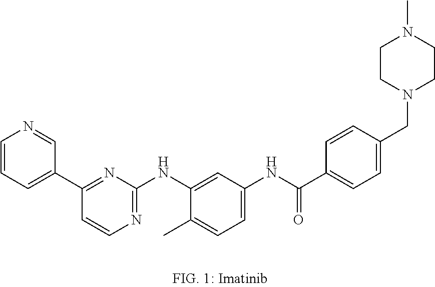 [SMILES: Cc1ccc(NC(=O)c2ccc(CN3CCN(C)CC3)cc2)cc1Nc1nccc(-c2cccnc2)n1]

Imatinib is available in tablet form (Gleevec®) of 100 mg and 400 mg for the treatment of Philadelphia chromosome positive chronic myeloid leukemia, Philadelphia chromosome positive acute lymphoblastic leukemia, myelodysplastic/myeloproliferative diseases, mastocytosis, hypereosinophilic syndrome (HES) and/or chronic eosinophilic leukemia, dermatofibrosarcoma protuberans and gastrointestinal stromal tumors.

EP0564409 patent discloses the preparation of imatinib and the use thereof especially as an antitumor agent.

EP1501485 patent relates to tablets comprising a pharmacologically effective amount of imatinib or a pharmaceutically acceptable salt thereof in an amount from 30% to 80% in weight of the active moiety based on the total weight of the tablet.

TR2008/02061 patent application relates to tablets comprising imatinib or a pharmaceutically acceptable salt thereof, a hydrate thereof or a salt of hydrate thereof in an amount above 80% in weight of the active moiety based on the total weight of the tablet, together with pharmaceutically acceptable excipients.

EP2723322 patent relates to an oral pharmaceutical composition, such as a tablet, including greater than 80% of Imatinib by weight based on the total weight of the composition and the process for preparation thereof.

EP2068835 patent application relates to solid solutions comprising imatinib and a solid solvent. Solid solution of said invention is then granulated, granules so-obtained are mixed with at least one excipient to obtain final mixture, said final mixture is tableted and optionally coated.

EP2120877 patent application describes a solid dispersion of imatinib mesylate comprising imatinib mesylate and a pharmaceutically acceptable carrier, wherein said carrier is a cellulose derivative.

EP2782560 patent application discloses a pharmaceutical powder formulation comprising granules of a tyrosine kinase inhibitor, wherein the granules of the tyrosine kinase inhibitor are coated with an enteric coating, wherein the tyrosine kinase inhibitor is present in an amount of up to 23% by weight based on the total weight of the pharmaceutical powder formulation.

EP3019159 patent application related to a granulate composition comprising 90-99.95% w/w of Imatinib mesylate and processes for preparation thereof, composition may be useful in the treatment of cancer.

WO2014041551 patent discloses oral aqueous solution comprising Imatinib or pharmaceutically acceptable acid addition salts or polymorphs thereof, process for preparing such solution and their use in the treatment of chronic myeloid leukemia, gastrointestinal stromal tumours. Further patent discloses oral aqueous solution which essentially comprises viscosity regulating agent that is used to stabilize the active ingredient (i.e. Imatinib mesylate) or increase the viscosity of the oral solution.

WO2019021229 patent discloses liquid dosage forms of protein-tyrosine kinase inhibitor such as Imatinib or pharmaceutically acceptable salt thereof. In particular, the patent relates to ready to use, liquid dosage forms of Imatinib or pharmaceutically acceptable salt thereof and to the processes for the preparation thereof.

The approved Imatinib tablet (Gleevec®) label dosage and administration discloses that for patients who are unable to swallow the film-coated tablets, the tablets may be dispersed in a glass of water or apple juice. The required number of tablets should be placed in the appropriate volume of beverage (approximately 50 mL for a 100 mg tablet, and 200 mL for a 400 mg tablet) and stirred with a spoon. The suspension should be administered immediately after complete disintegration of the tablet.

There are various disadvantages associated with such an administration, wherein Imatinib (Gleevec®) tablets take much longer time to get dispersed in water or apple juice and it leaves behind lots of residues in the container. Further, Imatinib has bitter taste and administration with apple juice may mask its taste and increase the palatability and patient compliance. But apple juice or any other flavoured beverage may not be available all the time while administering a drug to the patient. Therefore, it will be desirable to have Imatinib containing dosages in liquid forms which also contain sweeteners and flavours which make such dosage forms palatable and more patient compliant. Further, liquid dosage forms provide assurance of dosage uniformity upon administration to patients and eliminate difficulty of administration. Liquid dosage forms can also provide physicians more flexibility in designing dosage regimens for patients. Such liquid dosage forms are advantageous to pediatric patients and those patients who are unable to take oral therapy.

The inventors of the present invention have found a simple alternate approach for solving above problems by developing a pharmaceutical composition comprising imatinib or its pharmaceutically acceptable salt thereof and one or more pharmaceutical acceptable excipients in powder form, which can be reconstituted with a diluent just before administration. The said pharmaceutical composition according to the present invention provides better patient compliance.

### OBJECT OF THE INVENTION

The primary object of the present invention is to provide a pharmaceutical composition comprising imatinib or its pharmaceutically acceptable salt thereof and one or more pharmaceutical acceptable excipients in powder form, which can be reconstituted with a diluent just before administration.

Another object of the present invention is to provide a pharmaceutical composition comprising imatinib or its pharmaceutically acceptable salt thereof and one or more pharmaceutical acceptable excipients in powder form, wherein imatinib is present in an amount more than 80% by weight based on the total weight of the powder formulation.

Another object of the present invention is to provide a pharmaceutical composition comprising imatinib or its pharmaceutically acceptable salt thereof and one or more pharmaceutical acceptable excipients in powder form, which can be reconstituted with a diluent just before administration, wherein the said composition is substantially free from any surfactant/stabilizer.

Another object of the present invention is to provide a pharmaceutical composition comprising imatinib or its pharmaceutically acceptable salt thereof and one or more pharmaceutical acceptable excipients in powder form, which can be reconstituted with a diluent just before administration, wherein the said composition is used for the treatment of cancer.

Another object of the present invention is to provide a pharmaceutical composition comprising imatinib or its pharmaceutically acceptable salt thereof and one or more pharmaceutical acceptable excipients in powder form, which can be reconstituted with a diluent just before administration, wherein the said composition is used for the treatment of cancer in pediatric patients.

Another object of the present invention is to provide a pharmaceutical composition comprising imatinib or its pharmaceutically acceptable salt thereof and one or more pharmaceutical acceptable excipients selected from one or more fillers, one or more binders, one or more sweetener, one or more flavoring agent, optionally one or more preservative or the mixtures thereof.

Another object of the present invention is to provide a pharmaceutical composition comprising imatinib or its pharmaceutically acceptable salt thereof and one or more pharmaceutical acceptable excipients in powder form, wherein the said composition does not have more than 3% (w/w) of total impurity of imatinib, more preferably does not have more than 1% of total impurity of imatinib, after being stored at specific storage conditions.

Another object of the present invention is to provide a pharmaceutical composition comprising imatinib or its pharmaceutically acceptable salt thereof and one or more pharmaceutical acceptable excipients in powder form, wherein the said composition does not have more than 0.3% (w/w) of unspecified impurity of imatinib, after being stored at specific storage conditions.

Another object of the present invention is to provide a process for preparation of a pharmaceutical composition comprising imatinib or its pharmaceutically acceptable salt thereof and one or more pharmaceutical acceptable excipients in powder form.

Another object of the present invention is to provide a process for preparation of a pharmaceutical composition comprising imatinib or its pharmaceutically acceptable salt thereof and one or more pharmaceutical acceptable excipients in powder form, which can be reconstituted with a diluent just before administration.

Another object of the present invention is to provide a kit comprising a pharmaceutical composition of imatinib or its pharmaceutically acceptable salt thereof and one or more pharmaceutical acceptable excipients in powder form and a diluent, wherein the said pharmaceutical composition is reconstituted with a diluent just before administration.

### SUMMARY OF THE INVENTION

The present invention relates to a pharmaceutical composition comprising imatinib or its pharmaceutically acceptable salt thereof and one or more pharmaceutical acceptable excipients in powder form, which can be reconstituted with a diluent just before administration. Further, the present invention provides process for the preparation of the said composition and its use for the treatment of cancer in pediatric patients.

### DETAILED DESCRIPTION

Unless otherwise indicated, terms in this specification are intended to have their ordinary meaning in the relevant art.

The present invention related to a pharmaceutical composition comprising imatinib or its pharmaceutically acceptable salt thereof and one or more pharmaceutical acceptable excipients in powder form, which can be reconstituted with a diluent just before administration.

The term “Imatinib” used throughout the specification refers to not only their base per se, but also their other pharmaceutically acceptable salt, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof, wherein the amount of imatinib is present from 50 mg to 2000 mg in composition.

The term “pharmaceutically acceptable” means salt, carriers, excipients, and other formulation ingredients that are compatible with all other pharmaceutical ingredients of a composition and are not deleterious to an individual treated with composition.

The term “stable” as used throughout the specification, refers to a pharmaceutical composition in which the active pharmaceutical ingredients imatinib is present in an amount of at least 90% of the original label specified amount for each such ingredient during at least 3 months at 40° C./75% RH (i.e. Relative humidity).

The term “specific storage conditions” as used throughout the specification, refers to the pharmaceutical composition of present invention stored for at least 3 months at 40° C./75% RH.

The term “total impurities” of imatinib as used throughout the specification, refers to identified or unidentified degradation product or impurity structurally related with imatinib which are arising from a manufacturing process or during storage of material at specific storage conditions.

The term “unspecified impurity” of imatinib as used throughout the specification refers to can be either any unidentified impurity which is arising from a manufacturing process or during storage of material at specific storage conditions.

In one of the embodiments, the present invention is to provide a pharmaceutical composition comprising imatinib or its pharmaceutically acceptable salt thereof and one or more pharmaceutical acceptable excipients in powder form, wherein imatinib is present in an amount more than 80% by weight based on the total weight of the powder formulation, more preferably imatinib is present in an amount from 80% to 95% by weight based on the total weight of the powder formulation., most preferably imatinib is present in an amount from 80% to 90% by weight based on the total weight of the powder formulation.

In one of the another embodiments, the present invention is to provide a pharmaceutical composition comprising imatinib or its pharmaceutically acceptable salt thereof and one or more pharmaceutical acceptable excipients in powder form, which can be reconstituted with a diluent just before administration, wherein the said composition is substantially free from any surfactant/stabilizer.

The term “substantially free of surfactant/stabilizer” means the said pharmaceutical composition comprises less than about 1%, more preferably less than about 0.5%, most preferably less than about 0. 1% of surfactant/stabilizer by total weight of the composition.

In another embodiment, the present invention is to provide a pharmaceutical composition comprising imatinib or its pharmaceutically acceptable salt thereof and one or more pharmaceutical acceptable excipients in powder form, which can be reconstituted with a diluent just before administration, wherein the said composition is used for the treatment of cancer.

In another embodiment, the present invention is to provide a pharmaceutical composition comprising imatinib or its pharmaceutically acceptable salt thereof and one or more pharmaceutical acceptable excipients in powder form, which can be reconstituted with a diluent just before administration, wherein the said composition is used for the treatment of cancer in pediatric patients.

In another embodiment, the pharmaceutical composition according to the present invention can be used for single-dose or multi-dose administration to adults as well as to pediatric patients.

In another embodiment, the present invention is to provide a pharmaceutical composition comprising imatinib or its pharmaceutically acceptable salt thereof and one or more pharmaceutical acceptable excipients in powder form, which can be reconstituted with a diluent just before administration, wherein the said composition can be used for multi-dose administration for the treatment of cancer in pediatric patients.

In yet another embodiment, according to the present invention, the reconstitution time required to dissolve the pharmaceutical composition in a diluent is less than about 5 minutes, and more preferably less than 3 minutes. The reconstitution would require gentle mixing to provide a solution, which can be administered to the patient.

In yet another embodiment, the present invention is to provide a pharmaceutical composition comprising imatinib or its pharmaceutically acceptable salt thereof and one or more pharmaceutical acceptable excipients selected from one or more fillers, one or more binders, one or more sweetener, one or more flavoring agent, optionally one or more preservative or the mixtures thereof. The amount of each excipient in a powder formulation may vary within ranges conventional in the art.

The fillers can be selected from the group comprising of but not limited to mannitol, dibasic calcium phosphate anhydrous, microcrystalline cellulose, corn starch, sucrose or other sugar or sugar derivatives, low substituted HPC, pregelatinized starch or mixture thereof. The fillers can be present in a concentration of from about 10% to about 40% by weight of the total weight of the composition.

The binder can be selected from the group comprising of but not limited to pregelatinized starch, polyvinylpyrrolidone, povidone, copovidone, hydroxypropyl methyl cellulose, hydroxypropyl cellulose, maize starch, microcrystalline cellulose or mixture thereof. The binder can be present in a concentration of from about 0.5% to about 5% by weight of the total weight of the composition.

The sweetener can be selected from the group comprising of but not limited glucose, sucralose, trehalose, fructose, xylose, dextrose, galactose, tagatose, maltose, sucrose, glycerol, dulcitol, mannitol, lactitol, sorbitol, xylitol, saccharine and the like or any combinations thereof. The Sweetener can be present in a concentration of from about 0.01% to about 5% by weight of the total weight of the composition.

The flavoring agent can be selected from the group comprising of but not limited forest berry, synthetic flavor oils and flavoring aromatics and/or natural oils, extracts from plants leaves, flowers, fruits, and so forth and the like or any combinations thereof. The flavoring agent can be present in a concentration of from about 0.01% to about 5% by weight of the total weight of the composition.

The diluent can be selected from the group comprising of but not limited purified water, fruit juice, or syrup mixture thereof. The diluent can be used to reconstitute the powder form.

The preservative can be selected from the group comprising of but not limited alcohol, benzalkonium chloride, benzethonium chloride, benzoic acid, benzyl alcohol, boric acid, bronopol, butylene glycol, butylparaben, calcium acetate, calcium chloride, calcium lactate, cetrimide, cetylpyridinium chloride, chlorhexidine, chlorobutanol, chlorocresol, chloroxylenol, citric acid mono- hydrate, cresol, glycerin, hexetidine, imidurea, methyl paraben, mono-thioglycerol, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propylene glycol, propylparaben, propylparaben sodium, sodium acetate, sodium benzoate, sodium borate, sodium lactate, sodium metabisulfite, sodium propionate, sodium sulfite, sorbic acid, sulfur dioxide, thimerosal. More preferably, the said preservatives is methyl paraben and propyl paraben.

The solvent can be selected from the group comprising of but not limited purified water, ethanol, isopropanol, acetone or mixture thereof. The solvent can be used during the granulation stage as a granulating fluid.

The pharmaceutical composition described herein may be prepared by conventional technology well known to those skilled in the art, such as wet granulation, dry granulation and dry compaction methods and the like thereof.

The present invention is to provide a process for preparation of a pharmaceutical composition comprising imatinib or its pharmaceutically acceptable salt thereof and one or more pharmaceutical acceptable excipients in powder form comprising following steps.
a) Blending imatinib with filler to obtain a dry mix.
b) Preparing the granulating fluid by mixing the binder, sweetener, flavoring agent with solvent.
c) Granulating the dry mix material using the granulating fluid.
d) Drying the granulated mass to obtain granules.

In yet another embodiment, the present invention is to provide a process for preparation of a pharmaceutical composition comprising imatinib or its pharmaceutically acceptable salt thereof and one or more pharmaceutical acceptable excipients in powder form comprising following steps.
a) Blending Imatinib with mannitol to obtain dry mix.
b) Preparing the granulating fluid by mixing the solvents isopropyl alcohol and water, and adding hypromellose, sucralose and forest berry flavor in the solvent mixture to get clear solution.
c) Granulating the dry mix material using granulating fluid.
d) Drying the granulated mass to obtain dried granules.

In yet another embodiment, the present invention is to provide a process for preparation of a pharmaceutical composition comprising imatinib or its pharmaceutically acceptable salt thereof and one or more pharmaceutical acceptable excipients in powder form, which can be reconstituted with a diluent just before administration comprising following steps.
a) Blending Imatinib with mannitol to obtain dry mix.
b) Preparing the granulating fluid by mixing the solvents isopropyl alcohol and water, and adding hypromellose, sucralose and forest berry flavor in the solvent mixture to get clear solution.
c) Granulating the dry mix material using granulating fluid.
d) Drying the granulated mass to obtain dried granules and optionally lubricating the granules to obtain a blend of lubricated granules.
e) Filling the required quantity of blend of lubricated granules in one of the chambers and purified water as diluent in another chamber of the dual chambered kit using Powder filling machine and a continuous motion liquid filling line with capper.

In yet another embodiment, the present invention is to provide a process for preparation of a pharmaceutical composition comprising imatinib or its pharmaceutically acceptable salt thereof and one or more pharmaceutical acceptable excipients in powder form, which can be reconstituted with a diluent just before administration comprising following steps.
a) Blending Imatinib with mannitol to obtain dry mix.
b) Preparing the granulating fluid by mixing the solvents isopropyl alcohol and water, and adding hypromellose in the solvent mixture to get clear solution.
c) Granulating the dry mix material using granulating fluid.
d) Drying the granulated mass to obtain dried granules and optionally lubricating the granules to obtain a blend of lubricated granules.
e) To prepare suspending vehicle by mixing sucralose with forest berry flavor and purified water.
f) Filling the required quantity of blend of lubricated granules in one of the chambers and suspending vehicle in another chamber of the dual chambered kit using Powder filling machine and a continuous motion liquid filling line with capper.

In yet another embodiment, the present invention is to provide a process for preparation of a pharmaceutical composition comprising imatinib or its pharmaceutically acceptable salt thereof and one or more pharmaceutical acceptable excipients in powder form, which can be reconstituted with a diluent just before administration comprising following steps.
a) Blending Imatinib with mannitol to obtain dry mix.
b) Preparing the granulating fluid by mixing the solvents isopropyl alcohol and water, and adding hypromellose in the solvent mixture to get clear solution.
c) Granulating the dry mix material using granulating fluid.
d) Drying the granulated mass to obtain dried granules and optionally lubricating the granules to obtain a blend of lubricated granules.
e) To prepare suspending vehicle by mixing sucralose, methyl paraben, propyl paraben with forest berry flavor and purified water.
f) Filling the required quantity of blend of lubricated granules in one of the chambers and suspending vehicle in another chamber of the dual chambered kit using Powder filling machine and a continuous motion liquid filling line with capper.

In yet another embodiment, the present invention is to provide a kit comprising a pharmaceutical composition of imatinib or its pharmaceutically acceptable salt thereof and one or more pharmaceutical acceptable excipients in powder form and a diluent, wherein the said pharmaceutical composition is reconstituted with a diluent before administration.

The term “kit” refers to a packaging system comprising at least of dual chambers, wherein one chamber comprises the pharmaceutical composition and another chamber comprises diluent. During the time of administration of the said pharmaceutical composition, the contents of both the chambers of the kit are mixed together to obtain a reconstituted solution comprising the said pharmaceutical composition with the diluent.

The term “powder form” refers to type of preparations including powders, granules, beads and the likes thereof. These types of preparations are convenient to dispense in a kit or a dual chambered component, such that the pharmaceutical composition containing the powder form of imatinib or its pharmaceutically acceptable salt thereof can be dispensed in one compartment and the diluent in the other compartment of the kit or a dual chambered component. Before administration, the powder form is mixed with the diluent to obtain a reconstituted solution comprising the said pharmaceutical composition with the diluent.

One of the examples of the kit is a dual chamber packaging system comprising a bottle with a cap, wherein the pharmaceutical composition is kept in a chamber in the cap, and the diluent is present in the bottle. During administration, the cap of the bottle is twisted to tighten the cap which causes the chamber in the cap to break or puncture and releases the pharmaceutical composition in the bottle comprising the diluent. By gentle shaking the contents of the pharmaceutical composition get reconstituted with the diluent. The cap of the bottle is opened, and the reconstituted composition is administered to the patient conveniently.

It is within the scope of the present invention to use the bottles of varying shapes, size and mechanisms of reconstituting the powder blend with the diluent. Further, other similar packaging containers can also be used such that it allows reconstitution of the powder blend with a diluent, and the oral solution can be administered to a patient. The present invention is not limited to a dual-chamber bottle, and the equivalent packaging containers are included within the scope of the invention.

In yet another embodiment, the present invention is to provide a pharmaceutical composition comprising imatinib or its pharmaceutically acceptable salt thereof and one or more pharmaceutical acceptable excipients in powder form, wherein the said composition does not have more than 3% (w/w) of total impurity of imatinib, more preferably does not have more than 1% of total impurity of imatinib, after being stored at specific storage conditions.

In yet another embodiment, the present invention is to provide a pharmaceutical composition comprising imatinib or its pharmaceutically acceptable salt thereof and one or more pharmaceutical acceptable excipients in powder form, wherein the said composition does not have more than 0.3% (w/w) of unspecified impurity of imatinib, after being stored at specific storage conditions.

### EXAMPLE

The present invention has been described by way of example only. It is to be recognized that modifications falling within the scope and spirit of the claims, which would be obvious to a person skilled in the art based upon the disclosure herein, are also considered to be included within the scope of this invention. The scope of the invention is in no manner limited by the disclosed example.

### Example 1

### Pharmaceutical Composition of Imatinib

| | Strengths | | |
|---|---|---|---|
| | 150 mg/ | 300 mg/ | 1200 mg/ |
| Ingredients | 15 ml | 15 ml | 15 ml |
| Imatinib | 179.25 | 358.5 | 1434.0 |
| Mannitol | 32.83 | 65.66 | 262.64 |
| Hypromellose | 4.8 | 9.6 | 38.4 |
| Sucralose | 2.4 | 4.8 | 19.2 |
| Forest berry flavor | 0.72 | 1.44 | 5.76 |
| Total Blend | 220.0 | 440.0 | 1760.0 |
| Suspending Vehicle | | | |
| Purified Water | QS to 15 ml | QS to 15 ml | QS to 15 ml |
| Packaging component | | | |
| Dual Chamber PET bottle | 1 No. | 1 No. | 1 No. |
| Powder filling Kit | 1 No. | 1 No. | 1 No. |

### Manufacturing Process

a) Blending Imatinib with mannitol to obtain dry mix.
b) Preparing the granulating fluid by mixing the solvents isopropyl alcohol and water, and adding hypromellose, sucralose and forest berry flavor in the solvent mixture to get clear solution.
c) Granulating the dry mix material using granulating fluid.
d) Drying the granulated mass to obtain dried granules and optionally lubricating the granules to obtain a blend of lubricated granules.
e) Filling the required quantity of blend of lubricated granules in one of the chambers and purified water as diluent in another chamber of the dual chambered kit using Powder filling machine and a continuous motion liquid filling line with capper.

### STABILITY STUDY

TABLE 1
Stability data of imatinib powder for solution
of example 1 at condition of 40° C./75% RH.

| Stability time period | Unspecified impurity | Total impurity |
|---|---|---|
| Initial | 0.103 | 0.301 |
| 3 months | 0.144 | 0.486 |

The above data shows a total impurity not more than 3% of imatinib in the formulation indicative of stability of imatinib powder for solution in the drug product.

### Example 2

### Pharmaceutical Composition of Imatinib (Unit Dose)

| | Strengths | | |
|---|---|---|---|
| | 150 mg/ | 300 mg/ | 1200 mg/ |
| Ingredients | 15 ml | 15 ml | 15 ml |

Powder Component (mg/Unit)

| | | | |
|---|---|---|---|
| Imatinib | 179.25 | 358.50 | 1434.00 |
| Mannitol | 15.95 | 31.90 | 127.60 |
| Hypromellose | 4.80 | 9.60 | 38.40 |
| Total Powder Blend | 200.00 | 400.00 | 1600.00 |
| Weight | | | |

Suspending Vehicle (mg/Unit)

| | | | |
|---|---|---|---|
| Sucralose | 2.40 | 4.80 | 19.20 |
| Flavor (forest Berry) | 0.72 | 1.44 | 5.76 |
| Purified Water | q.s. to 15 mL | q.s. to 15 mL | q.s. to 15 mL |

Packaging component

| | | | |
|---|---|---|---|
| Dual Chamber PET | 1 No. | 1 No. | 1 No. |
| bottle | | | |
| Powder filling Kit | 1 No. | 1 No. | 1 No. |

### Manufacturing Process

a) Blending Imatinib with mannitol to obtain dry mix.
b) Preparing the granulating fluid by mixing the solvents isopropyl alcohol and water, and adding hypromellose in the solvent mixture to get clear solution.
c) Granulating the dry mix material using granulating fluid.
d) Drying the granulated mass to obtain dried granules and optionally lubricating the granules to obtain a blend of lubricated granules.
e) To Prepare suspending vehicle by mixing sucralose with forest berry flavor and purified water.
f) Filling the required quantity of blend of lubricated granules in one of the chambers and suspending vehicle in another chamber of the dual chambered kit using Powder filling machine and a continuous motion liquid filling line with capper.

### STABILITY STUDY

TABLE 2
Stability data of 150 mg/15 imatinib powder (Unit
Dose) of example 2 at condition of 40° C./75% RH.

| Related | Stability | 40° C./75% RH | | | |
|---|---|---|---|---|---|
| substances | Limits | Initial | 30 Days | 90 Days | 180 Days |
| Unspecified | NMT 0.3% | 0.137 | 0.169 | 0.162 | 0.137 |
| Impurity | | | | | |
| Total Impurities | NMT 3% | 0.265 | 0.305 | 0.286 | 0.259 |
| Assay | 90-110% | 100.8 | 102.3 | 102.4 | 100.7 |

The above data shows a total impurity not more than 3% of imatinib in the formulation and the assay of imatinib is in range of 90-110%, indicative of stability of imatinib powder.

TABLE 3
Stability data of reconstituted 150 mg/15 ml imatinib powder for
solution (Unit Dose) of example 2 at condition of 40° C./75%

| | | 40° C./75% RH | | | |
|---|---|---|---|---|---|
| Related substances | Stability Limits | Initial | 30 Days | 60 Days | 90 Days |
| Unspecified | NMT 0.3% | 0.176 | 0.181 | 0.181 | 0.135 |
| impurity | | | | | |
| Total Impurities | NMT 3% | 0.315 | 0.327 | 0.318 | 0.267 |

The above data shows a total impurity not more than 0.6% of imatinib in the formulation indicative of stability of reconstituted imatinib powder for solution in the drug product.

### Example 3

### Pharmaceutical Composition of Imatinib with Preservative (Unit Dose)

| | Strengths | | |
|---|---|---|---|
| | 150 mg/ | 300 mg/ | 800 mg/ |
| Ingredients | 15 ml | 15 ml | 15 ml |

Powder Component (mg/Unit)

| | | | |
|---|---|---|---|
| Imatinib | 179.25 | 358.50 | 1434.00 |
| Mannitol | 15.95 | 31.90 | 127.60 |
| Hypromellose | 4.80 | 9.60 | 38.40 |
| Total Powder Blend | 200.00 | 400.00 | 1600.00 |
| Weight | | | |

Suspending Vehicle (mg/Unit)

| | | | |
|---|---|---|---|
| Sucralose | 2.40 | 4.80 | 19.20 |
| Flavor (forest Berry) | 0.72 | 1.44 | 5.76 |
| Methyl paraben | 3.66 | 7.31 | 19.5 |
| Propyl paraben | 0.28 | 0.56 | 1.50 |
| Purified Water | q.s. to 15 mL | q.s. to 15 mL | q.s. to 15 mL |

Packaging component

| | | | |
|---|---|---|---|
| Dual Chamber PET | 1 No. | 1 No. | 1 No. |
| bottle | | | |
| Powder filling Kit | 1 No. | 1 No. | 1 No. |

### Manufacturing Process

a) Blending Imatinib with mannitol to obtain dry mix.
b) Preparing the granulating fluid by mixing the solvents isopropyl alcohol and water, and adding hypromellose in the solvent mixture to get clear solution.
c) Granulating the dry mix material using granulating fluid.
d) Drying the granulated mass to obtain dried granules and optionally lubricating the granules to obtain a blend of lubricated granules.
e) To prepare suspending vehicle by mixing sucralose, methyl paraben, propyl paraben with forest berry flavor and purified water.
f) Filling the required quantity of blend of lubricated granules in one of the chambers and suspending vehicle in another chamber of the dual chambered kit using Powder filling machine and a continuous motion liquid filling line with capper.

### STABILITY STUDY

TABLE 4
Stability data of 150 mg/15 ml Imatinib Powder
for solution with Preservative (Unit Dose strategy)
of example 3 at condition of 40° C./75% RH.

| Related | Stability | 40° C./75% RH | | | |
|---|---|---|---|---|---|
| substances | Limits | Initial | 30 Days | 60 Days | 90 Days |
| Unspecified | NMT 0.2% | 0.152 | 0.152 | 0.153 | 0.130 |
| impurities | | | | | |
| Total Impurities | NMT 3% | 0.269 | 0.270 | 0.272 | 0.242 |
| Assay | 90-110% | 102.7 | 101.8 | 103.8 | 101.3 |

The above data shows a total impurity not more than 3% of imatinib in the formulation, and the assay of imatinib is in range of 90-110%, indicative of stability of imatinib powder for solution in the drug product.

The stability data as mentioned above indicate that the pharmaceutical composition comprising imatinib or its pharmaceutically acceptable salt thereof and one or more pharmaceutical acceptable excipients in powder form reconstituted with a diluent just before use are stable.

Further, the pharmaceutical composition in the powder form gets reconstituted in the diluent within 3 minutes of gentle mixing.

### Example 4

### Pharmaceutical Composition of Imatinib with Preservative (Multi-Dose)

| | Strengths | | |
|---|---|---|---|
| | | 400 mg/ | 400 mg/ |
| | 800 mg/ | 5 mL; | 5 mL; |
| Ingredients | 15 mL | 40 mL | 70 mL |

Powder Component (mg/Unit)

| | | | |
|---|---|---|---|
| Imatinib | 956 | 3824.00 | 6692.00 |
| Mannitol | 85.1 | 340.27 | 595.47 |
| Hypromellose | 25.6 | 102.40 | 179.20 |
| Isopropanol | Qs | Qs | Qs |
| Purified Water | Qs | Qs | Qs |
| Total Powder Blend Weight | 1066.7 | 4266.67 | 7466.67 |

Suspending Vehicle (mg/Unit)

| | | | |
|---|---|---|---|
| Sucralose | 12.8 | 51.20 | 89.60 |
| Flavor (forest Berry) | 3.84 | 15.36 | 26.88 |
| Methyl paraben | 19.5 | 78.00 | 136.50 |
| Propyl paraben | 1.5 | 6.00 | 10.50 |
| Purified Water | Qs to 15 ml | Qs to 40 ml | Qs to 70 ml |

Packaging component

| | | | |
|---|---|---|---|
| Dual Chamber PET bottle | 1 No. | 1 No. | 1 No. |
| Powder filling Kit | 1 No. | 1 No. | 1 No. |

The oral compositions of the present invention can be administered as multi-dose formulations to pediatrics patients.

We claim:

1. A pharmaceutical composition comprising imatinib or its pharmaceutically acceptable salt and one or more pharmaceutical acceptable excipients in powder form which can be reconstituted with a diluent just before administration.

2. The pharmaceutical composition according to claim 1, wherein the imatinib is present in an amount more than 80% by weight based on the total weight of the powder formulation.

3. The pharmaceutical composition according to claim 1, wherein the said composition is substantially free from any surfactant/stabilizer.

4. The pharmaceutical composition according to claim 1, wherein the said composition is used for the treatment of cancer in adults as well as pediatric patients.

5. The pharmaceutical composition according to claim 1, wherein the said composition can be used as single-dose or multi-dose administration to adults as well as pediatric patients

6. The pharmaceutical composition according to claim 1, wherein one or more pharmaceutically acceptable excipients selected from one or more fillers, one or more binders, one or more sweetener, one or more flavoring agent or the mixtures thereof and optionally one or more preservative.

7. A process for preparation of a pharmaceutical composition comprising imatinib or its pharmaceutically acceptable salt and one or more pharmaceutical acceptable excipients in powder form comprising the steps of:
a) Blending imatinib with filler to obtain a dry mix.
b) Preparing the granulating fluid by mixing the binder, sweetener, flavoring agent with solvent.
c) Granulating the dry mix material using the granulating fluid.
d) Drying the granulated mass to obtain granules.

8. A process for preparation of a pharmaceutical composition comprising imatinib or its pharmaceutically acceptable salt and one or more pharmaceutical acceptable excipients in powder form, which can be reconstituted with a diluent just before administration comprising the steps of:
a) Blending imatinib with filler to obtain a dry mix
b) Preparing the granulating fluid by mixing the binder, sweetener, flavoring agent with solvent.
c) Granulating the dry mix material using the granulating fluid.
d) Drying the granulated mass to obtain granules and optionally lubricating the granules to obtain a blend of lubricated granules.
e) Filling the required quantity of blend of lubricated granules in one of the chambers and purified water as diluent in another chamber of the dual chambered kit using Powder filling machine and a continuous motion liquid filling line with capper.

9. The pharmaceutical composition according to claim 1, wherein the reconstitution time required to dissolve the pharmaceutical composition in a diluent is less than 5 minutes.

10. The pharmaceutical composition according to claim 1, wherein the said composition does not have more than 3% w/w of total impurity of imatinib and assay of imatinib is in range of 90-110% after being stored at 40° C./75% RH for at least 3 months.
